(19) United States
(12) Patent Application Publication
(10) Pub. No.: US 2014/0121743 A1
(43) Pub. Date: May 1, 2014

(54) HOUSING CONSTRUCTIONS WITH INCREASED IMPACT RESISTANCE

(71) Applicants: Charles Roger Aaron Leigh, North Epping NSW (AU); Kirsten Tran, Kensington NSW (AU)

(72) Inventors: Charles Roger Aaron Leigh, North Epping NSW (AU); Kirsten Tran, Kensington NSW (AU)

(21) Appl. No.: 13/664,410

(22) Filed: Oct. 30, 2012

(51) Int. Cl. A61N 1/05; H05K 13/00; H05K 5/02

(52) U.S. Cl. CPC A61N 1/0504; H05K 5/02; H05K 13/0023
USPC 607/137; 361/679.01; 29/592.1

(57) ABSTRACT

Housing constructions as disclosed herein can be an implantable component of a medical device and are designed to provide an enhanced degree of protection to electrical and/or mechanical components disposed therein to impact forces onto the housing from an external object to thereby increase the effective service life of devices making use of such housing constructions.

### FIELD

Housing constructions and methods for making the same and as disclosed herein display improved properties of impact resistance, thereby affording an enhanced degree of protection to electrical and/or mechanical components disposed therein.

### BACKGROUND

Housing constructions are used for a variety of different end-use device applications. Certain devices that include electrical and/or mechanical components typically make use of a housing for the purpose of enclosing the electrical and/or components, thereby protecting them from damage as such electrical and/or mechanical components can be relatively fragile and can be damaged easily. Such damage can be caused by impact from an object and/or by exposure to a particular environment, e.g., a high moisture environment. Housings conventionally operate to both enclose the device electrical and/or mechanical components and protect them from such types of damage.

Devices that make use of such housings include all types of consumer electronic devices such as cellular phones, portable music players, and the like. Additionally, medical devices that include electrical and/or mechanical components make use of housings, and such housings may be configured for placement outside of a user's body or for placement within a user's body, e.g., can be an implantable housing. Examples of medical devices comprising such housings include those used for treating certain heart conditions, hearing loss conditions, or the like. Hearing prosthesis make use of components that include housings that, depending on the particular application, may be used outside of the user's body or that may be implanted within the user's body.

While such housings may be configured to provide a certain resistance to mild levels of impact, such housings are not able offer a level of impact resistance useful to protect the enclosed electrical and/or mechanical components from damage in the event of a large impact, or doing so would require an undesirable increase in the thickness of the device housing. It is, therefore, desired to provide a housing construction that is designed in a manner to provide a greater level of impact resistance than provide by conventional housing constructions to thereby offer an enhanced degree of protection to electrical and/or mechanical components disposed therein, thereby increasing the service life of devices comprising the same in the event of experiencing a large impact.

### SUMMARY

Housing constructions as disclosed herein can be provided as part of a medical device, wherein such housing can be closed or hermetically sealed and comprise one or more components disposed therein that can be sensitive to impact damage, such as electrical and/or mechanical components. The housing can be implantable into a user's body, and be an implantable component of a hearing prosthesis, such as a cochlear implant and the like.

The housing construction can include a volume of a substantially incompressible fluid positioned adjacent a wall surface of the housing. The substantially incompressible fluid can be selected from the group consisting of Newtonian fluids, shear-thickening fluids, shear-thinning fluids, thixotropic fluids, pseudoplastic fluids, and combinations thereof. In an example, the volume of incompressible fluid can be disposed within a cavity of a member that is positioned outside of the housing adjacent a housing external surface. In another example, the enclosed volume of substantially incompressible fluid can be disposed within an internal cavity inside the housing itself. In either case, the volume of incompressible fluid operates to protect the one or more components disposed within the housing from an impact force to the housing.

When the volume of incompressible fluid is disposed within an internal cavity of the housing, the volume preferably extends within the housing between opposed housing inside surfaces, wherein one of the housing inside surfaces is an inside surface of a housing exterior wall. In such example, the incompressible fluid can be in contact with or adjacent one or more of the components, or the housing internal cavity can include a region that does not include the incompressible fluid and wherein one or more of the components are disposed within such region. In this example, where the volume of incompressible fluid is disposed within the housing internal cavity, the volume of the incompressible fluid comprises at least 20 percent of a total volume of the internal cavity within the housing. The region not including the incompressible fluid can comprise a gas mixture disposed therein.

### BRIEF DESCRIPTION OF THE DRAWINGS

These and other features and advantages of housing constructions and methods for making the same, as disclosed herein, will be appreciated as the same becomes better understood by reference to the following detailed description, when considered in connection with the accompanying drawings.

FIG. 1 is a perspective view of a housing construction;

FIGS. 2a and 2b are cross-sectional side views of housing construction before and after being subjected to an impact force;

FIGS. 3a to 3e are cross-sectional views of different example embodiment housing constructions as disclosed herein;

FIG. 4 is a schematic view of a hearing prosthesis comprising an implantable component with a housing construction as disclosed herein;

FIG. 5 is a schematic view of the implantable component and housing construction of FIG. 4; and

FIGS. 6a to 6c are schematic views of different example methods of making housing constructions as disclosed herein.

### DETAILED DESCRIPTION

Housing constructions as disclosed herein are made in a manner that provides an improved level of protection to electrical and/or mechanical components or structures and other elements susceptible or sensitive to impact damage, e.g., integrated circuits, solder connections, braze connections, braze joints between ceramic feedthroughs and the housing, and the like, that are disposed within the housing from an impact force to the housing. Specifically, housing constructions are specially engineered to include a volume of a substantially incompressible medium that operates to buffer or absorb the shock associated with an external impact force to the housing, and/or resist the housing from being deformed or compressed due to an external impact, which thereby operates to protect the internal components from being damaged due to such impact force.

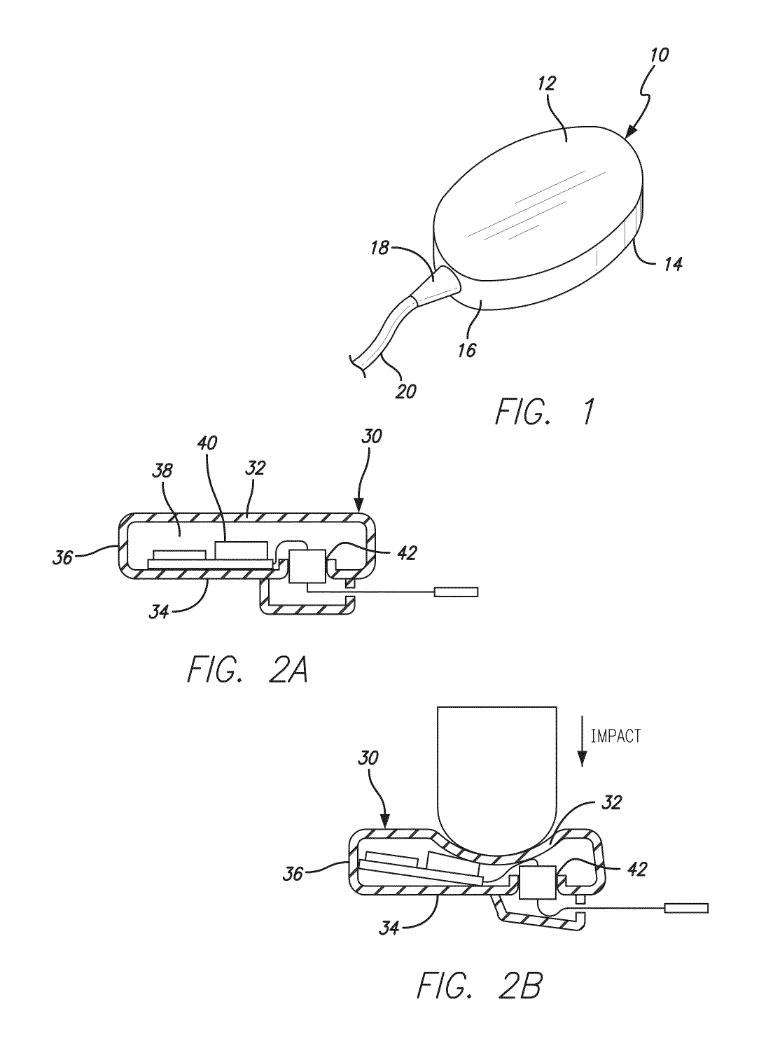

FIG. 1 illustrates a housing 10 comprising an external top surface 12, an external bottom surface 14, and an external side surface or external side wall 16 extending therebetween and defining an outer circumference of the housing. The housing also includes a feedthrough 18 extending through the housing 10 and configured to facilitate the passage of electrical signals between the electrical components and external components such as an actuator, electrode array, sensor or power source. The housing 10 can be configured as called for by the particular end-use application, and the embodiment illustrated in FIG. 1 is provided only for purposes of reference and example.

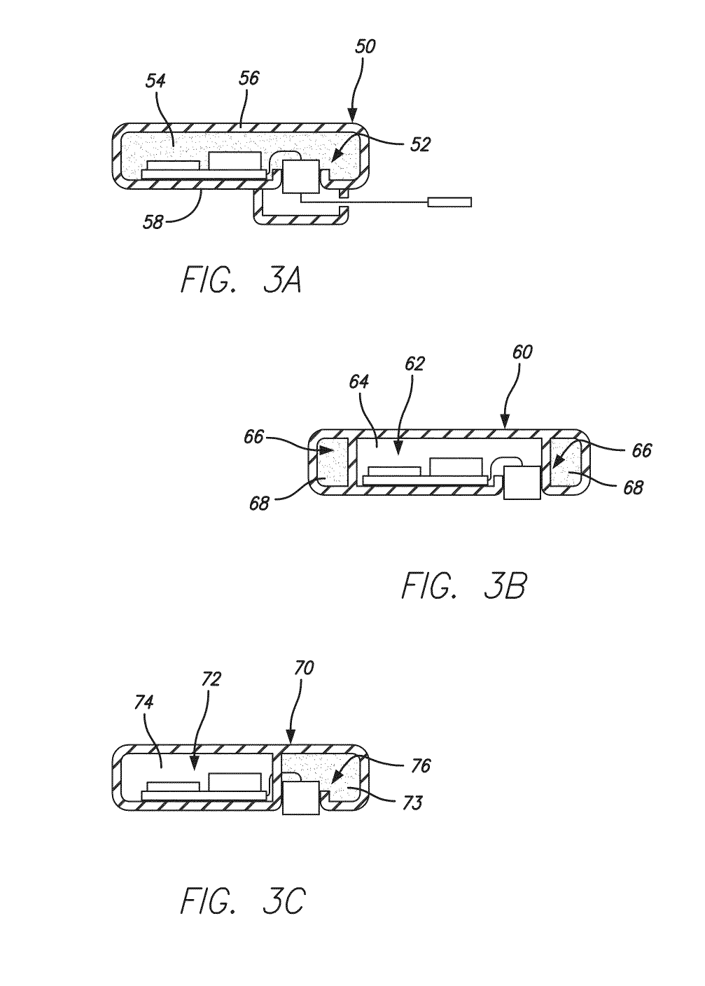

FIGS. 2a and 2b illustrate side views of a housing 30 in different states of existence. FIG. 2a illustrates the housing 30 in a normal state of existence, showing the top 32, bottom 34, and side surfaces 36, wherein each is in a normal undamaged or uncompressed state. The housing 30 includes an internal cavity 38, wherein electrical components 40 and/or mechanical components 42 are disposed therein. The housing can be made out of a variety of structural materials, e.g., plastic, metal, or the like, capable of providing a desired level of protection to the electrical and/or mechanical components enclosed therein.

The particular example illustrated includes mechanical components in addition to electrical components. Additionally, the housing 30 is illustrated as comprising an component or element extending outwardly therefrom and that is configured to provide a feature or function that is dependent on the device end-use application. As illustrated in FIG. 2a, the internal cavity 38 comprises a volume that is filled with air or gas or a gas mixture, depending on the particular end-use application. As illustrated, there is a sufficient gap or tolerance between the electrical components 40 and/or mechanical components 42 and an inside wall of the housing external top surface 32 to provide an additional degree of protection to such components under normal circumstances, this in addition to protection afforded by the structure of the housing itself.

FIG. 2b illustrates the housing 30 in an exemplary state of existence after it has been subjected to an impact force from an external object. As illustrated, an impact force directed onto the housing external top surface 30 has caused the top surface to compress and deform inwardly towards the bottom surface 34 a sufficient amount to contact the electrical components 40 and/or mechanical components 42 disposed therein, thereby causing one or more of the components to be damaged. Additionally, shock forces from the impact can cause one or more of the components to be damaged independent of any damage caused by directed contact with the deformed housing.)

A feature of housing constructions as disclosed herein is that they are specially engineered to provide an improved level of resistance to external impact forces to better protect the internal components disposed therein from impact forces (as depicted in FIG. 2b), thereby effectively extending the effective service life of devices that make use of such housing constructions. Housing constructions are developed to protect components disposed therein from damage which includes impact to electrical, mechanical and hermeticity performance. Housing constructions as disclosed herein are engineered to include a volume of a substantially incompressible medium, e.g., a fluid, positioned adjacent an external wall of the housing to resist damage induced by impact and/or and compression of the housing due to an external impact force and/or to absorb the force of the same to minimize or eliminate shock forces that may travel to the internal components. As illustrated in the different examples provided below, the volume of the incompressible medium can be positioned differently inside or outside of the housing, differently within the housing, and/or the extent or volume of the incompressible medium that is present can and will vary.

As used herein, the term “external wall” of the housing is understood to include not only the external surface of the housing but may include other wall surfaces of the housing that may not be the external wall, e.g., where the housing includes layers of materials forming walls or the like disposed above and/or below the external wall.

Materials useful for serving as the incompressible medium for use in this regard include non-Newtonian fluids, Newtonian fluids, and combinations thereof. Newtonian fluids are defined as materials which in smooth, uniform flow exhibit a linear relationship in its stress-strain curve, meaning its viscosity remains constant at different shear rates. Non-Newtonian fluids are fluids that do not display a linear relationship at different shear rates, so that its viscosity can vary when a different shear stress is applied. In an example embodiment, non-Newtonian fluids, e.g., shear-thickening, can be useful to provide a shock absorbing feature within the housing as the shock of an impact can be dissipated across the volume of the non-Newtonian fluid when it changes in viscosity, which can operate to protect the relatively delicate electronic and/or mechanical components within the housing. Time independent non-Newtonian fluids can be used in these applications because the duration of shear stress due to a single impact force is relatively miniscule so that time may not need to be considered as a function.

Materials useful as the incompressible medium can include shear-thickening or dilatant fluids, which increase in viscosity with an increase stress, and thixotropic and pseudoplastic fluids, which decrease in viscosity with increased stress. Other materials that can be used here when the housing construction is an implantable component of a medical device include biocompatible materials such as silicone oils and the like. Typically a material with a relatively high viscously is preferred. In applications where the incompressible medium is placed into contact with electrical components it is desired that the incompressible medium be electrically nonconductive. Additionally, if desired, the incompressible medium can be selected to have other properties, such as being a moisture absorber or an absorber of other materials that can operate to impair proper operation of the components within the housing. Also, the incompressible material can be selected so that it has a self-sealing property in the event that the housing becomes damaged or otherwise hermeticity is breached so it can operate to hermetically or otherwise seal to increase the service life or safety or operation of the device.

While many examples of materials useful as an incompressible medium have been disclosed, it is to be understood that such examples are intended to be representative of the many types of incompressible materials that can be used, and are not intended to be limiting as to the specific incompressible material useful for making housing constructions as disclosed herein.

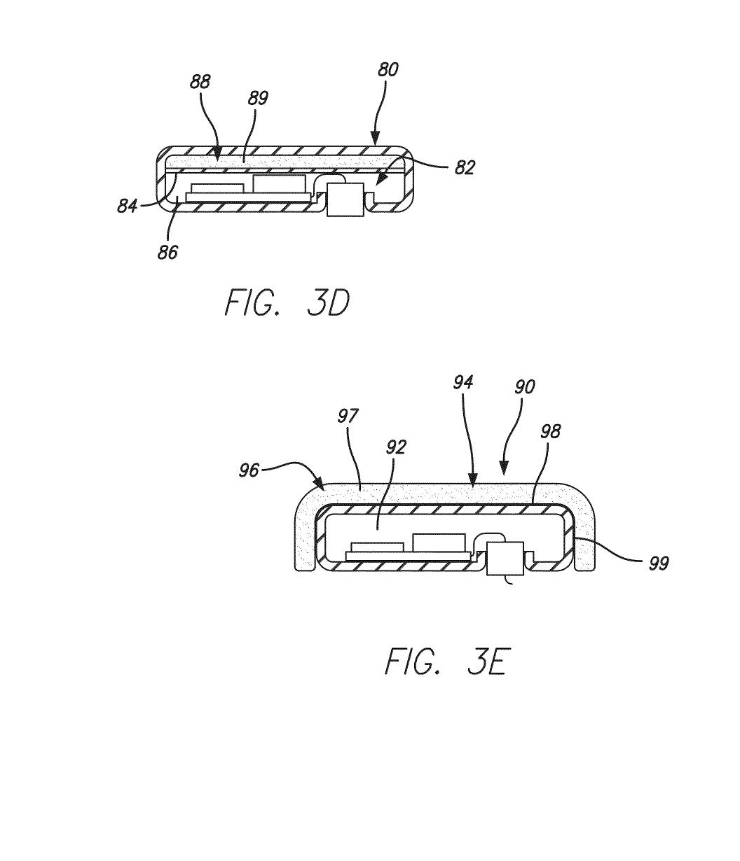

FIGS. 3a to 3d illustrate different example embodiment housing constructions as disclosed herein comprising different volumes and/or placement locations of the incompressible medium. Generally, it is desired that the incompressible medium be positioned within the housing at a location designed to take advantage of the incompressible characteristic of the medium to thereby minimize the structural compressive deformation of the housing in response to an external impact force. Further, it is desired that the internal cavity inside the housing where the incompressible material is disposed is one having a closed or fixed volume so as to maximize the advantage of the incompressible characteristic of the incompressible material to resist impact and compression of the housing caused thereform.

FIG. 3a illustrates an example embodiment housing construction 50 comprising the same general features as the housing noted in the example illustrated in FIG. 2b, except that the internal cavity 52 is filled with a volume of the incompressible medium 54. In such example, the volume of the internal cavity 32, extending between inside wall surfaces of the external top and bottom surfaces 56 and 58 of the housing is filled with the incompressible medium. In such example embodiment, the incompressible medium can be any of those disclosed above. A feature of this example is that the incompressible material extends between the opposed inside wall surfaces of the external top and bottom surfaces so as to provide an optimum resistance to an external impact onto one of external the top or bottom surfaces in a housing placement situation where the other of the external top or bottom surface is positioned against a rigid member. An example of such a placement position is one where the housing construction is an implantable component of a medical device that is positioned in the user's body with the external top surface positioned under the user's skin and the external bottom surface positioned against the user's bone. A further feature of this example is it can be easy implemented into use with existing housings with little or no modifications, e.g., by simply introducing the incompressible medium into the housing internal cavity.

FIG. 3b illustrates an example embodiment housing construction 60 comprising the same general features as the housing noted in the example illustrated in FIG. 2b, except that the internal cavity 62 has been specially designed to have more than one region or portion. Specifically, the cavity 62 now comprises a first region or portion 64 that is positioned centrally within the housing and that is filled with air or gas, and a second region or portion 66 that extends concentrically around the central portion and that is filled with a volume of the incompressible medium 68. Gas may be provided in the cavity first region for the purpose of providing an indication that a leak exists in the housing, e.g., when the housing is tested for being hermetically sealed. A feature of this example is that the internal cavity 62 has been specially engineered to isolate the incompressible medium from both the electrical components and mechanical components, i.e., such components are disposed within the cavity first region 64, while at the same time placing a sufficient volume of the incompressible material in an isolated cavity region or portion 66 that is designed to provide a desired level of impact resistance to the housing from an external impact to protect the components disposed within the housing from such impact force. Again, as with the example illustrated in FIG. 3a, the volume of incompressible medium extends within the housing between inside wall surfaces of the external top and bottom surfaces to provide a desired level of impact resistance when the housing is subjected to an impact force and positioned in the manner described above for FIG. 3a. While a particular placement location for the incompressible material-containing cavity region has been disclosed and illustrated, it is to be understood that other placement positions can and will exist depending on such factors as the placement position of the components inside of the housing, and number of components disposed within the housing, and the particular end-use application. For example, the electrical and/or mechanical components can be positioned along the side walls of the housing and the incompressible medium can be disposed within a region of the internal cavity that is disposed centrally within the housing (as opposed to being within an outer region of the cavity).

FIG. 3c illustrates an example embodiment housing construction 70 comprising the same general features as the housing noted in the example illustrated in FIG. 2b, except that the internal cavity 72 has been specially designed to have more than one portion or region. Specifically, the internal cavity 72 is now substantially bisected into a first region or portion 74 that is filled with air or gas, and a second region or portion 76 that is filled with a volume of the incompressible medium 78. This example embodies the same features as disclosed above for the example illustrated in FIG. 3b. An additional feature of this particular embodiment is that the volume of the incompressible material is further reduced as a function of the reduced volume of the cavity second region 76, and the placement of the cavity second region 76 may be located to where an impact event to the housing is likely to occur, or a less robust region of the medical device. In this example, the cavity region 76 containing the incompressible material may extend around more than about ¼, ½, or ¾ of the housing as needed to provide a desired level of impact resistance for a particular end-use application. In an example, the cavity portion containing the incompressible material extends about half way around the housing.

FIG. 3d illustrates an example embodiment housing construction 80 comprising the same general features as the housing noted in the example illustrated in FIG. 2b, except that the internal cavity 82 has been specially designed to have more than one region or portion. Specifically, the internal cavity 82 now includes a fixed partition 84 extending parallel to the housing external top and bottom surfaces and interposed therebetween. The partition 84 defines a cavity lower portion 86 that is filled with air or gas, and a cavity upper portion 88 that is filled with a volume of the incompressible medium 89. The electronic and/or mechanical components are disposed in the lower portion 86 and are isolated from the incompressible medium. The upper portion 88 comprises a volume of the incompressible medium. A feature of this embodiment is that it isolates the incompressible medium from the electronic and/or mechanical components, while also providing a degree of impact resistance to the housing in end-use applications where external impact forces are directed onto the housing external top surface. While the volume of the incompressible medium does not extend completely to the inside wall of the external bottom surface, a sufficient volume of the incompressible fluid is provided and positioned in such a manner so as to provide a sufficient level of protection to the internal electrical and/or mechanical components from an impact force onto the housing for use in certain end-use applications.

A feature of the four example housing constructions described above and illustrated in FIGS. 3a to 3d is the placement of the incompressible medium within and filling or substantially filling the internal cavity of the housing, or a region thereof, to provide a fixed, confined or enclosed volume of the incompressible medium. As used herein, the term “substantially” is understood to mean that some free space, e.g., comprising gas and/or internal components and not comprising the incompressible medium, may exist in the cavity or cavity region containing the incompressible medium while still permitting the incompressible medium to perform in the manner intended to protect the internal components from damage from impact. The larger the amount of free space not comprising the incompressible medium may mean that a greater deformation of the housing may occur before the incompressible fluid provides its intended protection from impact forces. In an example embodiment, it is desired that the housing internal cavity comprise about 20 percent by volume or less of free space, wherein the free space is measured as being the total internal cavity volume minus the volume occupied by the incompressible fluid and minus the volume occupied by the housing internal components. Ultimately, the amount of incompressible medium disposed within the housing can and will vary on such factors as the shape of the housing, the type of material and thickness of the same used to form the housing, and the particular end-use application and possible impact forces that the housing may be subjected to

FIG. 3e illustrates an example embodiment housing construction 90 comprising the same general features as the housing noted in the example illustrated in FIG. 2b, wherein the internal cavity 92 comprises an air or gas-filled chamber comprising electrical and/or mechanical components disposed therein. Additionally, this housing construction 90 comprises a member 94 that is external to the housing and is positioned adjacent at least a portion of one or more external surfaces of the housing, and that is constructed comprising a cavity 96 filled with the incompressible material 97. This example provides a feature of the incompressible material being outside of the housing, while providing a desired level of impact resistance to the housing to thereby protect the electronic and/or mechanical components disposed within the housing from damage. In this example, the member 94 is configured such that it extends along the housing external top surface 98 and down along the housing external side walls 99. Configured in this matter, the member 94 operates from outside of the housing to protect the housing from an impact force directed onto the member. Alternatively, the member 94 could also be disposed along only one surface of the housing and/or along only a portion thereof. A feature of example illustrated in FIG. 3e is that use of the member 94 enables easy retrofitability with existing housings with little or no modification. In an example embodiment, the member 94 can be attached to an external surface of the housing by conventional methods such as by welded, braze joint, adhesive, bolted, riveted attachment or the like. Alternatively the member 94 may not be rigidly attached to the housing but be held in position thereagainst by an interference fit provided by complementing mating interface surfaces of the member and housing. In this case, retrofitability could extend to fitting the additional impact protection member to an already implanted housing. While the example construction of FIG. 3e illustrates the member covering a particular portion of the housing, it is to be understood that the member can be configured to cover different and/or partial external surfaces of the housing as called for by the specific end-use applications, and that all such variations are understood to be within the scope of the construction as disclosed herein.

While the external member 94 has been disclosed as being used to provide a degree of impact protection to a housing and the impact-sensitive components disposed therein, the member can also be configured to provide a degree of impact protection to components other than housing that may also be otherwise susceptible to impact damage and benefit from protection. For example, the external member can be configured to protect leads and/or cables extending from the housing, wherein such external member can be part of or extend from an existing external member protecting the housing, or such external member can exist separate and apart from any external member protecting the housing.

Housing constructions as disclosed herein may be used in a variety of end-use applications. An example of one such application is where the housing construction is part of a medical device that may or may not be implanted into a user's body. Examples of such medical device applications include hearing prosthesis, heartbeat regulation devices, muscular tissue stimulation devices, neurological stimulation devices, and the like. In an example embodiment, housing constructions as disclosed herein can be used as an implantable component of a hearing prosthesis.

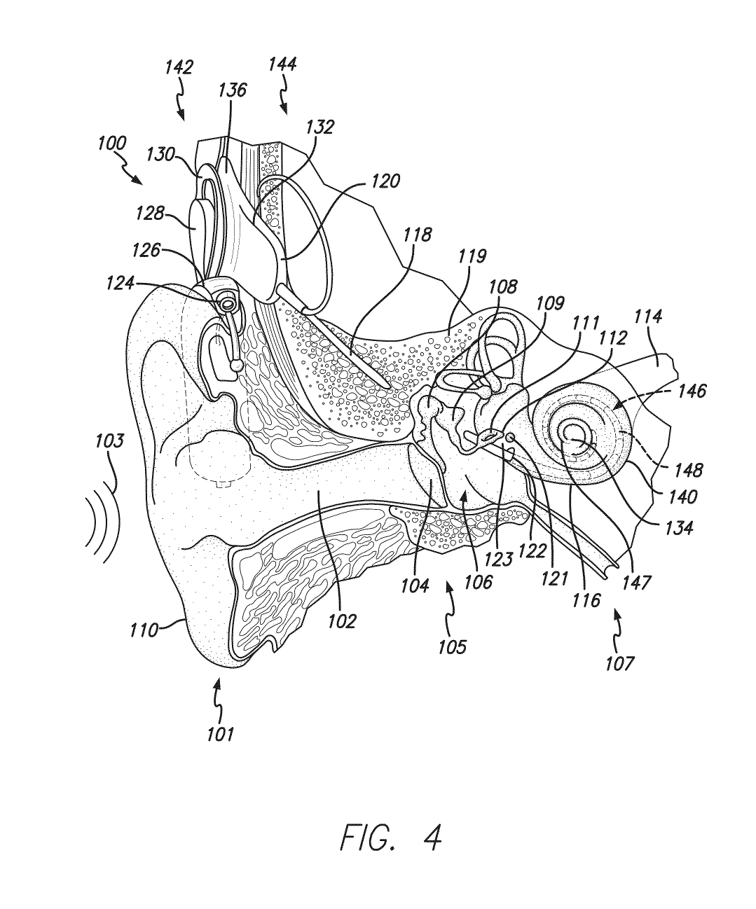

FIG. 4 illustrates a hearing prosthesis in the form of a cochlear implant system 100 that includes an internal component 144 typically having an internal receiver/transceiver unit 132, a stimulator unit 120, and an elongate stimulating assembly 118 comprising the electrode construction as disclosed herein. The internal receiver/transceiver unit 132 permits the cochlear implant system 100 to receive and/or transmit signals to an external device 126 and includes an internal coil 136, and preferably, a magnet (not shown) fixed relative to the internal coil 136. Internal receiver unit 132 and stimulator unit 120 are hermetically sealed within a biocompatible housing, sometimes collectively referred to as a stimulator/receiver unit. The magnets facilitate the operational alignment of the external and internal coils, enabling internal coil 136 to receive power and stimulation data from external coil 130. Elongate stimulating assembly 118 has a proximal end connected to stimulator unit 220, and a distal end implanted in cochlea 140. Stimulating assembly 118 extends from stimulator unit 120 to cochlea 140 through mastoid bone 119. In certain examples, external coil 130 transmits electrical signals (e.g., power and stimulation data) to internal coil 136 via a radio frequency (RF) link, as noted above. Internal coil 136 is typically a wire antenna coil comprised of multiple turns of electrically insulated single-strand or multi-strand platinum or gold wire. The electrical insulation of internal coil 136 is provided by a flexible silicone molding (not shown). In use, implantable receiver unit 132 may be positioned in a recess of the temporal bone adjacent auricle 110 of the recipient. Various types of energy transfer, such as infrared (IR), electromagnetic, capacitive and inductive transfer, may be used to transfer the power and/or data from external device to cochlear implant.

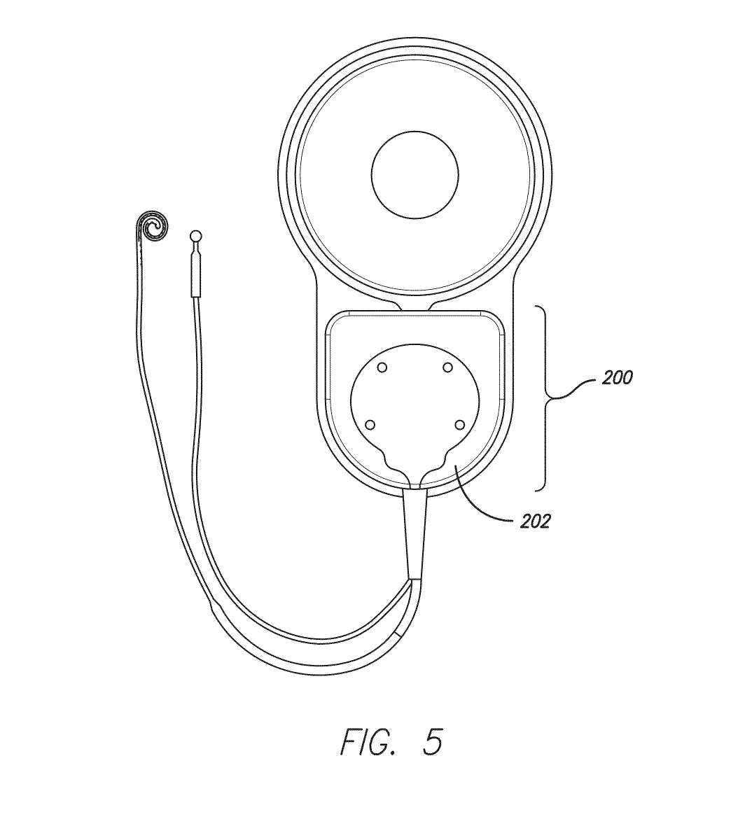

FIG. 5 illustrates the implanted components of the cochlear implant system of FIG. 5, and is provided for the purpose of specifically showing the stimulator/receiver unit 200 which is disposed within a housing construction 202 as disclosed herein. In this particular application, the housing construction 202 of the stimulator/receiver unit 200 is implanted and positioned beneath a user's skin, interposed between the skin and the skull. When implanted in this manner, the stimulator/receiver can be subjected to an external impact force, for example should the user slip or fall and hit their head in the location of where the stimulator/receiver unit housing is implanted.

In such an application, the housing construction is formed from a metallic material such as titanium or the like. Because of its implanted placement position, it is desired that the housing be made to provide a low profile fitting so as to not be visible. In such an application, it is difficult to gain the desired resistance to impact in the housing by simply increasing the thickness of the housing itself, as this will increase the size of the housing and the implanted profile. Thus, a feature of one or more of the housing constructions as disclosed herein is the ability to provide the desired increase in impact resistance without changing the external profile or size of the housing.

While the housing construction as discussed herein has been described and illustrated in FIGS. 4 and 5 as a component of a cochlear implant, it is to be understood that housing constructions can be used with other types of hearing prosthesis as a component that may or may not be implanted. Examples include but are not limited to bone conduction hearing prosthesis, wherein the housing construction is part of a behind the ear component and/or is part of an active implanted component.

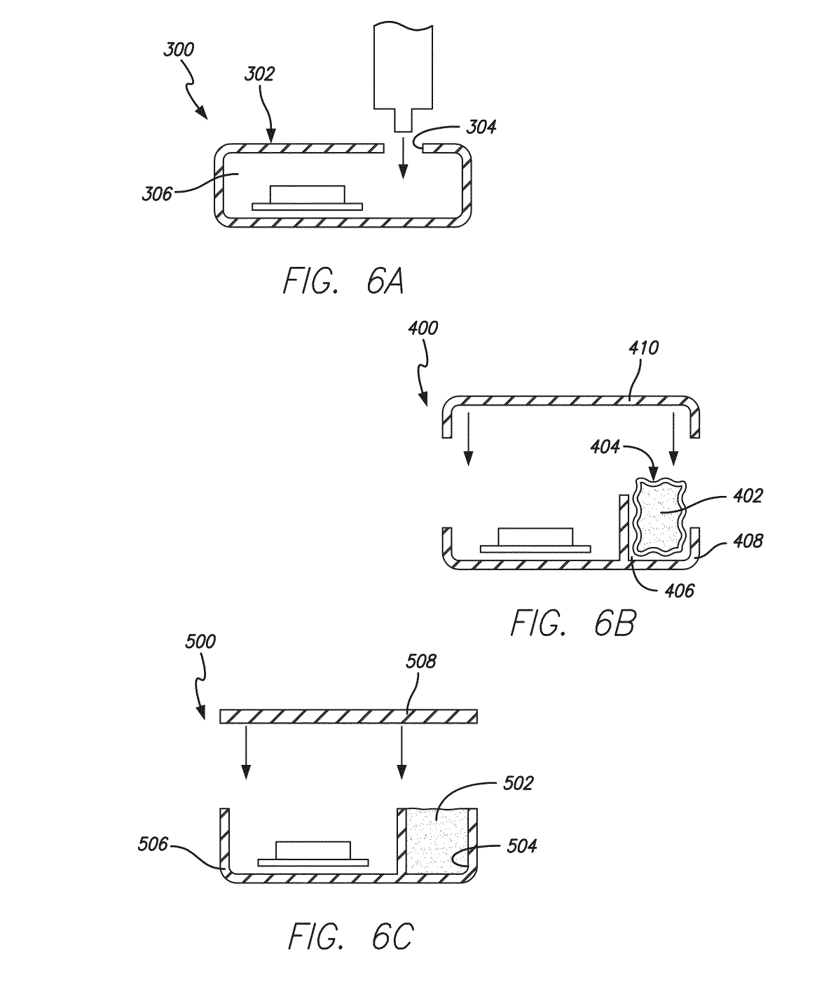

FIGS. 6a to 6c illustrate example methods that can be used to make housing constructions as disclosed herein. FIG. 6a illustrates a method of making 300 wherein the housing 302 and the elements disposed therein are assembled. The housing is either formed with an opening 304 or such opening is made in the housing, and the desired incompressible medium is introduced, for example by injection into the housing internal cavity 306, and the opening is sealed once the cavity is filled. A shear thinning non-Newtonian fluid can help facilitate the filling process as it will thin under the compressive forces applied during injection.

FIG. 6b illustrates a method or making 400 wherein the incompressible medium 402 is provided in a bladder 404 and the bladder 404 is positioned within a cavity portion 406 of a lower housing member 408 along with the remaining elements in the housing. An upper housing member or lid 410 is then positioned over the lower housing member and secured thereto to compress the bladder to fill the volume within the cavity portion.

FIG. 6c illustrates a method or making 500 wherein the incompressible medium 502 is disposed within a cavity portion 504 of a lower housing member 506 to fill it, along with the remaining elements in the housing, and an upper housing member or lid 508 is then positioned over the lower housing member and secured thereto to seal off the cavity portion. In an alternative embodiment the incompressible medium can be provided to the housing in solid form at assembly temperature that then forms a liquid at about 37° C.

These are understood to be but a few examples of how housing constructions as disclosed herein can be made, and it is to be understood that variations of these methods as well as alternatives of these methods can exist and that all such variations and/or alternatives are considered to be within the scope of making housing constructions as disclosed herein.

Certain example housing constructions and methods for making the same have been disclosed. While each such housing construction and method has been described with respect to a limited number of embodiments, the specific features of one embodiment housing construction should not be attributed to other embodiments of the housing construction. No single embodiment is representative of all aspects of housing constructions and methods of making the same as disclosed herein. In some embodiments, the housing construction or method for making the same may comprise features or steps not mentioned herein.

Variations and modifications from the described embodiments exist. For example, housing construction as disclosed herein may include, in addition to the incompressible medium, a further shock absorbing member such as an elastomeric element or the like disposed within the housing. Wherein the incompressible medium and the elastomeric element can provide additive resistance to compressive impact and shock absorbing properties to the housing construction. The methods of making housing constructions are described herein as comprising a number of acts or steps. These steps or acts may be practiced in any sequence or order unless otherwise indicated. Finally, any number disclosed herein should be construed to mean approximate, regardless of whether the word “about” or “approximately” is used in describing the number. The appended claims intend to cover all those modifications and variations as falling within the scope of the electrode constructions and methods for making the same as disclosed herein.

What is claimed is:

1. A medical device comprising:
a closed housing comprising an internal cavity and one or more components disposed within the internal cavity; and
a volume of a substantially incompressible fluid positioned adjacent a surface of the housing, the volume of substantially incompressible fluid protecting the one or more components from damage caused by an impact force to the housing.

2. The medical device as recited in claim 1 wherein the volume of substantially
incompressible fluid is disposed within a cavity of an external member positioned outside of the housing and adjacent the surface of the housing that is an external surface.

3. The medical device as recited in claim 2 wherein the incompressible fluid substantially
fills the cavity of the external member.

4. The medical device as recited in claim 1 wherein the incompressible fluid substantially
fills at least a region of the internal cavity.

5. The medical device as recited in claim 4 wherein the region extends between opposed
housing inside surfaces.

6. The medical device as recited in claim 5 wherein one of the housing inside surfaces is an
inside surface of a housing exterior wall.

7. The medical device as recited in claim 4 wherein the internal cavity includes a second
region that is substantially free of the incompressible fluid.

8. The medical device as recited in claim 7 wherein the one or more components are
disposed within the region substantially free of the incompressible fluid.

9. The medical device as recited in claim 7 wherein the region that is substantially free of
the incompressible fluid comprises a gas mixture disposed therein.

10. The medical device as recited in claim 4 wherein the internal cavity comprises about 20 percent by volume or less free space.

11. The medical device as recited in claim 1 wherein the one or more components are selected from the group consisting of electrical component, mechanical components, and combinations of the same.

12. The medical device as recited in claim 1 wherein the incompressible fluid is disposed adjacent to the one or more of the components.

13. The medical device as recited in claim 1 wherein the housing is hermetically-sealed and implantable into a user's body.

14. The medical device as recited in claim 1 wherein the housing is a component of a hearing prosthesis.

15. The medical device as recited in claim 14 wherein the housing is a component of a cochlear implant.

16. The medical device as recited in claim 1 wherein the incompressible fluid is selected from the group consisting of Newtonian fluids, shear-thickening fluids, shear-thinning fluids, thixotropic fluids, pseudoplastic fluids, and combinations thereof.

17. An implantable component of a medical device comprising:
a hermetically-sealed housing comprising an internal cavity;
at least one impact-sensitive component disposed within the internal cavity; and
an incompressible fluid disposed within the internal cavity.

18. The implantable component as recited in claim 17 wherein the incompressible fluid is interposed between opposed surfaces within the housing.

19. The implantable component as recited in claim 18 wherein at least one of the opposed surfaces is an inside wall surface of a housing external surface.

20. The implantable component as recited in claim 17 wherein the incompressible fluid is provided as a closed volume within the internal cavity.

21. The implantable component as recited in claim 17 wherein the internal cavity further comprises a region that is free of the incompressible fluid and that includes a gas mixture.

22. The implantable component as recited in claim 17 wherein the incompressible fluid is adjacent the impact-sensitive component.

23. The implantable component as recited in claim 17 wherein the incompressible fluid is positioned within a central portion of the housing relative to an axis passing through the housing opposed top and bottom external surfaces.

24. The implantable component as recited in claim 17 wherein the incompressible fluid is positioned along at least a portion of a side wall of the housing that extends between the top and bottom external surfaces.

25. The implantable component as recited in claim 17 wherein the housing is part of a hearing prosthesis.

26. The implantable component as recited in claim 25 wherein the hearing prosthesis is a cochlear implant, and the housing is part of a receiver/transmitter/stimulator unit.

27. The implantable component as recited in claim 17 wherein the internal cavity includes a gas mixture disposed therein.

28. A hearing prosthesis comprising:
an external coil positioned outside of a user's body;
an internal component comprising an internal receiver/transmitter/stimulator unit, wherein the internal component is implanted within a user's body and comprises a hermetically-sealed housing that includes an impact-sensitive component disposed therein, the housing having an internal cavity; and
an incompressible fluid substantially filling at least a region of the internal cavity to protect the impact-sensitive component from an impact force to the housing from an external object.

29. The hearing prosthesis as recited in claim 28 wherein the internal cavity includes a region that is substantially free of the incompressible fluid and that includes a gas mixture.

30. The hearing prosthesis as recited in claim 28 wherein the incompressible fluid is adjacent the impact-sensitive component.

31. The hearing prosthesis as recited in claim 28 wherein the incompressible fluid extends between opposed surfaces within the housing, and wherein one of the opposed surfaces is an inside wall surface of a housing top or bottom external surface.

32. The hearing prosthesis as recited in claim 31 wherein the housing is implanted within the user's body with the top external surface positioned adjacent the user's skin, and with the bottom external surface positioned adjacent the user's skull.

33. A method for protecting an impact-sensitive component disposed within an implantable medical device housing from an external impact force, the method comprising the steps of:
forming a housing comprising at least one impact-sensitive component disposed therein; and
placing a volume of substantially incompressible fluid within an internal cavity, wherein the incompressible fluid is positioned adjacent a surface of the housing between a potential trajectory of an external impact causing object and the at least one impact-sensitive component.

34. The method as recited in claim 33 wherein during the step of forming, the using is made having the internal cavity disposed therein.

35. The method as recited in claim 34 wherein the incompressible fluid extends from at least one inside wall surface of a housing top or bottom external surface.

36. The method as recited in claim 34 wherein during the step of placing, the internal cavity comprises 20 percent by volume or more of free space.

37. The method as recited in claim 34 wherein during the step of forming, the internal cavity includes a region that is substantially free of the incompressible fluid and that includes a gas mixture.

38. The method as recited in claim 34 wherein during the step of placing, the incompressible fluid is position adjacent the at least one impact-sensitive component.

39. The method as recited in claim 33 wherein the internal cavity is disposed within an external member, and the external member is positioned adjacent the surface of the housing that is an external surface.
